(19) United States
(12) Patent Application Publication
(10) Pub. No.: US 2025/0144545 A1
(43) Pub. Date: May 8, 2025

(54) PURIFYING PRECURSOR COMPOUNDS AND RELATED SYSTEMS AND METHODS

(71) Applicant: ENTEGRIS, INC., Billerica, MA (US)

(72) Inventor: Peter Litak, Schwenksville, PA (US)

(21) Appl. No.: 18/937,784

(22) Filed: Nov. 5, 2024

Related U.S. Application Data
(60) Provisional application No. 63/547,452, filed on Nov. 6, 2023.

(51) Int. Cl. B01D 3/34; B01D 1/22

(52) U.S. Cl. CPC B01D 3/34; B01D 1/22

(57) ABSTRACT

Systems and methods for purifying precursor compounds are provided. A method comprises one or more of the following steps: mixing a crude product solution with a first solvent, wherein the crude product solution comprises a precursor compound and at least one impurity; wherein a boiling point of the first solvent is greater than a boiling point of the crude product solution; feeding a mixture of at least the crude product solution and the first solvent to an evaporator; and collecting, from the evaporator, a distillate comprising the precursor compound, wherein a purity of the precursor compound in the distillate is greater than 95% as determined by ¹H NMR. Other methods and systems are provided herein.

### CROSS-REFERENCE TO RELATED APPLICATIONS

This application claims the benefit under 35 USC 119 of U.S. Provisional Patent Application No. 63/547,452, filed Nov. 6, 2023, the disclosure of which is hereby incorporated herein by reference in its entirety.

### FIELD

The present disclosure relates to the field of distilling sensitive material from crude mixtures using high boiling point solvents and related systems and related methods.

### BACKGROUND

The distillation of sensitive materials from a crude mixture can be challenging due to the impurities found within the crude mixture, resulting in the yield loss of sensitive material.

### SUMMARY

Some embodiments of the present disclosure relate to a method comprising: mixing a crude product solution with a first solvent, wherein the crude product solution comprises a precursor compound and at least one impurity; wherein a boiling point of the first solvent is greater than a boiling point of the crude product solution; feeding a mixture of at least the crude product solution and the first solvent to an evaporator; and collecting, from the evaporator, a distillate comprising the precursor compound, wherein a purity of the precursor compound in the distillate is greater than 95% as determined by ¹H NMR.

Some embodiments relate to a system comprising a crude product solution comprising a precursor compound and at least one impurity; a first solvent having a boiling point that is greater than a boiling point of the crude product solution; and an evaporator, wherein, when a mixture of the crude product solution and the first solvent is fed to the evaporator, the evaporator is configured to product a distillate comprising the precursor compound, wherein a purity of the precursor compound in the distillate is greater than 95% as determined by ¹H NMR.

### DRAWINGS

Some embodiments of the disclosure are herein described, by way of example only, with reference to the accompanying drawings. With specific reference now to the drawings in detail, it is stressed that the embodiments shown are by way of example and for purposes of illustrative discussion of embodiments of the disclosure. In this regard, the description taken with the drawings makes apparent to those skilled in the art how embodiments of the disclosure may be practiced.

FIG. 1 illustrates a flowchart of a method for distilling a precursor compound from a crude mixture using high boiling point solvents, according to some embodiments.

FIG. 2 is a ¹H NMR spectra of the crude product solution and distillate fractions, according to some embodiments.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

Any prior patents and publications referenced herein are incorporated by reference in their entireties.

Throughout the specification and claims, the following terms take the meanings explicitly associated herein, unless the context clearly dictates otherwise. The phrases “in one embodiment,” “in an embodiment,” and “In some embodiments,” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

Some embodiments relate to methods for purifying precursor compounds and related systems, methods, and compositions. Isolating precursor compounds from crude product solutions can be difficult due to challenges associated with decomposition of the precursor compound (i.e., product), presence of undesirable decomposition reaction byproducts, and/or undesirable interaction of the reaction byproducts with the precursor compound. In addition, when isolating or purifying the precursor compound, various residues in form of oils and solids, among others, which cannot flow through a system used for isolating and/or purifying the precursor compound, decompose to form other byproducts and thus also introduce impurities. As disclosed herein, to overcome these and other challenges, in some embodiments, a high-boiling point co-solvent is introduced into the system. In some embodiments, the solvent can reduce solids deposition and formation, facilitate movement through the system of solid and viscous residues, and/or reduce or eliminate occurrence of undesirable reactions that produce undesirable byproducts. As disclosed herein, the solvent can result in improved yields and high purity levels, among numerous other advantages.

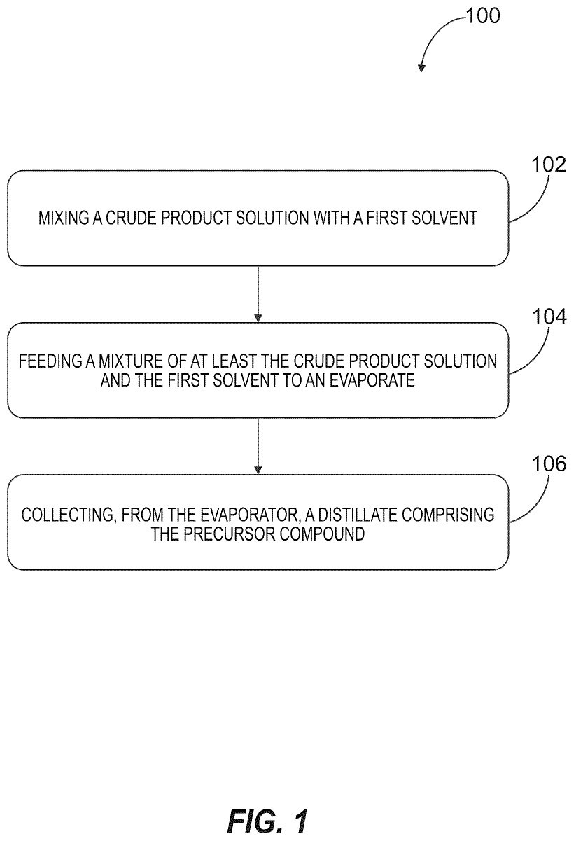

FIG. 1 is a flowchart of a method for separating a precursor compound from a crude product solution, according to some embodiments. As shown in FIG. 1, the method 100 for separating a precursor compound from a crude product solution may comprise one or more of the following steps: mixing 102 a crude product solution with a first solvent; feeding 104 a mixture of at least the crude product solution and the first solvent to an evaporator; and collecting 106, from the evaporator, a distillate comprising a precursor compound. Although not shown, in some embodiments, the method further comprises collecting a residue comprising the at least one impurity.

At step 102, in some embodiments, the method 102 comprises mixing a crude product solution with a first solvent.

In some embodiments, the mixing comprises contacting the crude product solution and the first solvent. In some embodiments, the mixing comprises bringing the crude product solution and the first solvent into immediate or close proximity. In some embodiments, the mixing comprises bringing the crude product solution and the first solvent into direct physical contact. In some embodiments, the mixing comprises agitating the crude product solution and the first solvent. In some embodiments, the mixing comprises stirring the crude product solution and the first solvent. In some embodiments, the mixing comprises combining the crude product solution and the first solvent. It will be appreciated that other techniques for mixing and/or contacting may be employed herein without departing from the scope of this disclosure.

In some embodiments, the first solvent is a high-boiling point solvent. In some embodiments, the first solvent is a co-solvent. In some embodiments, for example, the first solvent is a co-solvent because the crude product solution comprises at least one solvent. In some embodiments, for example, the crude product solution comprises a second solvent. In some embodiments, the crude product solution comprises a precursor compound. In some embodiments, the crude product solution comprises a precursor compound and at least one impurity. In some embodiments, the precursor compound is dissolved in the second solvent. In some embodiments, the at least one impurity is not dissolved in the second solvent. In some embodiments, the first solvent is inert with respect to the precursor compound. In some embodiments, the at least one impurity is at least partially dissolved in the second solvent. In some embodiments, at least a portion of the precursor compound is dissolved in the second solvent. In some embodiments, at least a portion of the precursor compound is not dissolved in the second solvent. In embodiments, the at least one first solvent may serve as a carrier to keep residues, viscous oils, reaction byproducts, and/or at least one impurity mobile in the crude product solution and/or mixture.

The first solvent may comprise a solvent having a boiling point that is greater than at least one of the crude product solution, the second solvent, or any combination thereof. In some embodiments, a boiling point of the first solvent is greater than a boiling point of the crude product solution. In some embodiments, the boiling point of the first solvent is at least 10%, at least 20%, at least 30%, at least 40%, at least 50%, at least 60%, at least 70%, at least 80%, at least 90% at least 100%, at least 200%, at least 300%, at least 400%, at least 500% greater than the boiling point of the crude product solution, or any range or subrange between 10% and 1000%.

In some embodiments, a boiling point of the first solvent is greater than a boiling point of the second solvent. In some embodiments, the boiling point of the first solvent is at least 10%, at least 20%, at least 30%, at least 40%, at least 50%, at least 60%, at least 70%, at least 80%, at least 90% at least 100%, at least 200%, at least 300%, at least 400%, at least 500% greater than the boiling point of the second solvent, or any range or subrange between 10% and 1000%.

In some embodiments, the boiling point of the first solvent is 250° C. to 500° C., or any range or subrange between 250° C. and 500° C. In some embodiments, the boiling point of the first solvent is 275° C. to 500° C., 300° C. to 500° C., 325° C. to 500° C., 350° C. to 500° C., 375° C. to 500° C., 400° C. to 500° C., 425° C. to 500° C., 450° C. to 500° C., 475° C. to 500° C., 250° C. to 475° C., 250° C. to 450° C., 250° C. to 425° C., 250° C. to 400° C., 250° C. to 375° C., 250° C. to 350° C., 250° C. to 325° C., 250° C. to 300° C., or 250° C. to 275° C.

In some embodiments, the boiling point of the crude product solution is lower than the boiling point of the at least one first solvent. In some embodiments, the boiling point of the crude product solution is 50° C. to 245° C., or any range or subrange between 50° C. and 245° C. In some embodiments, the boiling point of the crude product solution is 50° C. to 245° C., 50° C. to 240° C., 50° C. to 235° C., 50° C. to 230° C., 50° C. to 225° C., 50° C. to 220° C., 50° C. to 215° C., 50° C. to 210° C., 50° C. to 205° C., 50° C. to 200° C., 50° C. to 175° C., 50° C. to 150° C., 50° C. to 125° C., 50° C. to 100° C., 50° C. to 75° C.

As mentioned above, the first solvent can comprise solvent having a boiling point that is greater than a boiling point of the crude product solution and/or the second solvent; and/or that is inert with respect to the precursor compound. Accordingly, the first solvent may depend on the precursor compound. In some embodiments, a first solvent comprises an isoparaffinic hydrocarbon fluid. In some embodiments, the first solvent comprises at least one of a diphenyl ether fluid, a dimethyl sulfoxide fluid, a triethyl phosphate fluid, an isoparaffinic hydrocarbon fluid, or any combination thereof. It will be appreciated that other solvents can be used herein without departing from the scope of this disclosure.

As mentioned above, the second solvent can comprise a solvent in which the precursor compound is soluble and/or that has a boiling point that is less than a boiling point of the first solvent and/or a solvent in which the at least one impurity is not soluble. Examples of the second solvent include, without limitation, at least one of hydrocarbons, alkanes, alkenes, alkynes, cycloalkanes, aromatic hydrocarbons, heterocyclic compounds, or any combination thereof. It will be appreciated that other solvents can be used herein without departing from the scope of this disclosure.

In some embodiments, the precursor compound comprises at least one of a tin-containing compound, a niobium containing compound, a tungsten-containing compound, or any combination thereof. In some embodiments, the precursor compound comprises at least one of alkyl substituted tin compounds (e.g., a monoalkyl tin (II) compound, a monoalkyl tin (IV) compound, etc.), tin halides, tin oxides, tin hydroxides, alkyl substituted niobium compounds, niobium halides, niobium oxides, niobium hydroxides, tungsten halides, tungsten oxides, tungsten hydroxides, or any combination thereof. In some embodiments, the at least one tin-containing compound comprises at least one of tin(II) chloride (SnCl₂), tin(IV) chloride (SnCl₄), trimethyltin (Me₃Sn), triethyltin (Et₃Sn), tributyltin (Bu₃Sn), or any combination thereof. In some embodiments, the at least one niobium-containing compound comprises at least one of niobium(V) chloride (NbCl₅), niobium(V) oxychloride (NbOCl₃), niobium ethoxide (Nb(OC₂H₅)₅), niobium(V) pentafluoride (NbF₅), or any combination thereof. In some embodiments, the at least one tungsten-containing compound comprises at least one of tungsten hexachloride (WCl₆), tungsten hexafluoride (WF₆), tungsten hexaethoxide (W(OC₂H₅)₆), tungsten oxychloride (WOCl₄), or any combination thereof.

In some embodiments, the at least one impurity comprises a solid impurity. In some embodiments, the at least one impurity comprises at least one of a salt compound, a dialkyl compound, or any combination thereof. In some embodiments, the at least one impurity comprises at least one of a dialkyl tin compound, a dialkyl niobium compound, a dialkyl tungsten compound, or any combination thereof. In some embodiments, the at least one impurity comprises at least one solid impurity. In some embodiments, the at least one solid impurity comprises at least one salt compound or at least one dialkyl compound. In some embodiments, the at least one salt compound comprises at least one of a sodium chloride (NaCl), an ammonium chloride, or any combination thereof. In some embodiments, the at least one dialkyl compound comprises at least one of dimethyl sulfide, diethyl sulfide, dimethyl ether, diethyl ether, or any combination thereof.

At step 104, in some embodiments, the method 100 comprises feeding a mixture of the crude product solution and the first solvent to an evaporator.

In some embodiments, the feeding comprises flowing the mixture of the crude product solution and the first solvent to the evaporator. In some embodiments, the feeding comprises supplying the mixture of the crude product solution and the first solvent to the evaporator. In some embodiments, the feeding comprises introducing the mixture of the crude product solution and the first solvent to the evaporator. In some embodiments, the feeding comprises pumping the mixture of the crude product solution and the first solvent to the evaporator. In some embodiments, the feeding comprises introducing the mixture of the crude product solution and the first solvent in the evaporator. In some embodiments, the feeding comprises disposing the mixture of the crude product solution and the first solvent in the evaporator.

In some embodiments, the evaporator comprises at least one of a wiped film evaporator, a stirrer evaporator, a rising film evaporator, a circulation evaporator, a falling film evaporator, or any combination thereof. In some embodiments, the wiped film evaporator comprises concentrating, distilling, stripping, dehydrating, deodorization or any combination thereof.

In some embodiments, the mixture comprises 1% to 99% by volume of the first solvent based on the total volume of the mixture, or any range or subrange between 1% and 99%. In some embodiments, the mixture comprises 1% to 90%, 1% to 80%, 1% to 70%, 1% to 60%, 1% to 50%, 1% to 40%, 1% to 30%, 1% to 20%, 1% to 10%, 1% to 9%, 1% to 8%, 1% to 7%, 1% to 6%, 1% to 5%, 1% to 4%, 1% to 3%, 1% to 2%, 10% to 99%, 20% to 99%, 30% to 99%, 40% to 99%, 50% to 99%, 60% to 99%, 70% to 99%, 80% to 99%, or 90% to 99% by volume of the first solvent based on the total volume of the mixture.

In some embodiments, the mixture comprises 1% to 99% by volume of the crude product solution based on a total volume of the mixture, or any range or subrange between 1% and 99%. In some embodiments, the mixture comprises 1% to 98%, 1% to 97%, 1% to 96%, 1% to 95%, 1% to 94%, 1% to 93%, 1% to 92%, 1% to 91%, 1% to 90%, 1% to 80%, 1% to 70%, 1% to 60%, 1% to 50%, 1% to 40%, 1% to 30%, 1% to 20%, 1% to 10%, 10% to 99%, 20% to 99%, 30% to 99%, 40% to 99%, 50% to 99%, 60% to 99%, 70% to 99%, 80% to 99%, or 90% to 99% by volume of the crude product solution based on the total volume of the mixture.

In some embodiments, the mixture comprises 1% to 99% by volume of the second solvent based on a total volume of the mixture, or any range or subrange between 1% and 99%. In some embodiments, the mixture comprises 1% to 98%, 1% to 97%, 1% to 96%, 1% to 95%, 1% to 94%, 1% to 93%, 1% to 92%, 1% to 91%, 1% to 90%, 1% to 80%, 1% to 70%, 1% to 60%, 1% to 50%, 1% to 40%, 1% to 30%, 1% to 20%, 1% to 10%, 10% to 99%, 20% to 99%, 30% to 99%, 40% to 99%, 50% to 99%, 60% to 99%, 70% to 99%, 80% to 99%, or 90% to 99% by volume of the second solvent based on the total volume of the mixture.

At step 106, in some embodiments, the method 100 comprises collecting, from the evaporator, a distillate comprising the precursor compound.

In some embodiments, the collecting comprises recovering the distillate comprising the precursor compound in a flask or other container. In some embodiments, the collecting comprises disposing the distillate comprising the precursor compound in a collector. In some embodiments, the collector is under heating and/or cooling. It will be appreciated that other techniques for collecting the distillate may be employed without departing from the scope of this disclosure.

In some embodiments, prior to feeding a mixture of the crude product solution and the at least one first solvent to an evaporator, the precursor compound (e.g., in the crude product solution) has a purity of 95% or less as determined by ¹H NMR. In some embodiments, the precursor compound has a purity of less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 4%, less than 3%, less than 2%, less than 1%, less than 0.5%, less than 0.4%, less than 0.4% or less than 0.3%, less than 0.2%, less than 0.1% in the crude product solution. In some embodiments, prior to feeding a mixture of at least the crude product solution and the at least one first solvent to an evaporator, the precursor compound has a purity present in the crude product solution of 90% to 95% as determined by ¹H NMR. In some embodiments, the precursor compound has a purity of 91% to 95%, 92% to 95%, 93% to 95%, 94% to 95%, 94.9% to 95%, 94.99% to 95%, 94.999% to 95% in the crude product solution.

In some embodiments, a purity of the precursor compound in the distillate is greater than 95% as determined by ¹H NMR. That is, in some embodiments, upon collecting a distillate comprising a precursor compound, a purity of the precursor compound in the distillate is greater than 95% as determined by ¹H NMR. In some embodiments, the precursor compound has a purity of least 95%, at least 96%, at least 97%, at least 98%, at least 99%, at least 99.9%, at least 99.999%, at least 99.9999% as determined by ¹H NMR. In some embodiments, a purity of precursor compound in the distillate is 95% to 99.9999%, as determined by ¹H NMR, or any range or subrange between 95% and 99.9999%. For example, in some embodiments a purity of the precursor compound in the distillate is 95% to 96%, 95% to 97%, 95% to 98%, 95% to 99%, 95% to 99.9%, 95% to 99.99%, 95% to 99.999%, 95% to 99.9999%, 99.999% to 99.9999%, 99.99% to 99.9999%, 99.9% to 99.9999%, 99% to 99.9999%, 98% to 99.9999%, 97% to 99.9999%, 96% to 99.9999%, or 95% to 99.9999%, as determined by ¹H NMR.

Some embodiments relate to a system. In some embodiments, the system comprises a crude product solution, a first solvent, and an evaporator. In some embodiments, the crude product solution comprises at least one of a precursor compound, at least one impurity, a second solvent, or any combination thereof. In some embodiments, the first solvent has a boiling point that is greater than a boiling point of the crude product solution and/or second solvent. In some embodiments, the evaporator comprises at least one of wiped film evaporator, a stirrer evaporator, a rising film evaporator, a circulation evaporator, a falling film evaporator, or any combination thereof. In some embodiments, when a mixture of the crude product solution and the first solvent is fed to the evaporator, the evaporator is configured to produce a distillate comprising the precursor compound. In some embodiments, a purity of the precursor compound in the distillate is greater than 95% as determined by ¹H NMR. It will be appreciated that any of the crude product solutions, first solvents, evaporators, precursor compounds, at least one impurities, second solvents, etc. may be employed herein without departing from the scope of this disclosure.

### EXAMPLE

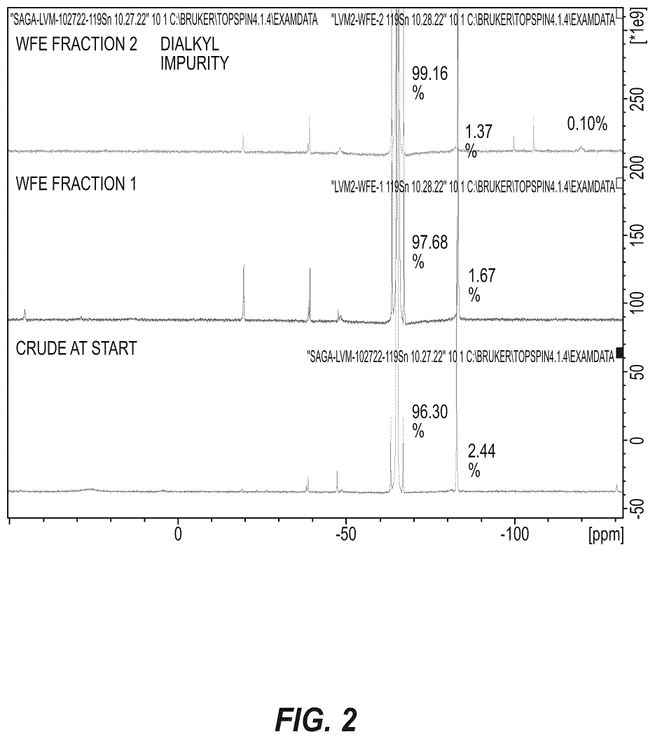

A crude product solution containing a tin-containing precursor compound was purified using a wiped film distillation system and process. The conditions included heating at 55° C. to 65° C. at 2.5 Torr at a distillation rate of 0.4 kg per hour. A 13% by volume of an isoparaffinic hydrocarbon fluid (Isopar V) was added to the crude product based on a total volume of the resulting mixture. The resulting ¹H NMR is presented in FIG. 2. As shown in FIG. 2, the wiped film distillation system and process produced a distillate comprising the tin-containing precursor compound at a purity of greater than 97% (WFE Fraction 1) and a purity of greater than 99% (WFE Fraction 2). Each WFE fraction had less dialkyl impurity than a comparative reboiler distillation. In addition, a rate of distillation was greater than the comparative reboiler distillation. These results indicated the addition of the isoparaffinic hydrocarbon fluid improved the purity of the tin-containing precursor compound in the distillate.

### ASPECTS

Various Aspects are described below. It is to be understood that any one or more of the features recited in the following Aspect(s) can be combined with any one or more other Aspect(s).

Aspect 1. A method comprising:
mixing a crude product solution with a first solvent,
   wherein the crude product solution comprises a precursor compound and at least one impurity;
   wherein a boiling point of the first solvent is greater than a boiling point of the crude product solution;
feeding a mixture of at least the crude product solution and the first solvent to an evaporator; and
collecting, from the evaporator, a distillate comprising the precursor compound,
   wherein a purity of the precursor compound in the distillate is greater than 95% as determined by ¹H NMR.

Aspect 2. The method according to Aspect 1, wherein the crude product solution further comprises a second solvent, wherein the precursor compound is dissolved in the second solvent.

Aspect 3. The method according to Aspect 2, wherein the at least one impurity is not dissolved in the second solvent.

Aspect 4. The method according to any one of Aspects 1-3, wherein a purity of the precursor compound in the crude product solution is 95% or less as determined by ¹H NMR.

Aspect 5. The method according to any one of Aspects 1-4, wherein a purity of the precursor compound in the crude product solution is 90% to 95% as determined by ¹H NMR.

Aspect 6. The method according to any one of Aspects 1-5, wherein the precursor compound comprises a tin-containing compound.

Aspect 7. The method according to any one of Aspects 1-6, wherein the precursor compound comprises a niobium-containing compound.

Aspect 8. The method according to any one of Aspects 1-7, wherein the precursor compound comprises a tungsten-containing compound.

Aspect 9. The method according to any one of Aspects 1-8, wherein the first solvent is inert with respect to the precursor compound.

Aspect 10. The method according to any one of Aspects 1-9, wherein the boiling point of the first solvent is at least 10% greater than a boiling point of the crude product solution.

Aspect 11. The method according to any one of Aspects 1-10, wherein the boiling point of the first solvent is 250° C. to 500° C.

Aspect 12. The method according to any one of Aspects 1-11, wherein the boiling point of the crude product solution is 50° C. to 200° C.

Aspect 13. The method according to any one of Aspects 1-12, wherein the first solvent comprises an isoparaffinic hydrocarbon fluid.

Aspect 14. The method according to any one of Aspects 1-13, wherein the at least one impurity comprises at least one solid impurity.

Aspect 15. The method according to any one of Aspects 1-14, wherein the at least one impurity comprises at least one of a salt compound, a dialkyl compound, or any combination thereof.

Aspect 16. The method according to any one of Aspects 1-15, wherein the purity of the precursor compound in the distillate is at least 99%.

Aspect 17. The method according to any one of Aspects 1-16, wherein the evaporator comprises at least one of a wiped film evaporator, a stirrer evaporator, a rising film evaporator, a circulation evaporator, a falling film evaporator, or any combination thereof.

Aspect 18. The method according to any one of Aspects 1-17, further comprising collecting a residue comprising the at least one impurity.

Aspect 19. A system comprising:
a crude product solution comprising a precursor compound and at least one impurity;
a first solvent having a boiling point that is greater than a boiling point of the crude product solution; and
an evaporator,
   wherein, when a mixture of the crude product solution and the first solvent is fed to the evaporator, the evaporator is configured to produce a distillate comprising the precursor compound,
   wherein a purity of the precursor compound in the distillate is greater than 95% as determined by ¹H NMR.

Aspect 20. The system according to Aspect 19, wherein the evaporator comprises at least one of wiped film evaporator, a stirrer evaporator, a rising film evaporator, a circulation evaporator, a falling film evaporator, or any combination thereof.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

What is claimed is:

1. A method comprising:
mixing a crude product solution with a first solvent,
wherein the crude product solution comprises a precursor compound and at least one impurity;
wherein a boiling point of the first solvent is greater than a boiling point of the crude product solution;
feeding a mixture of at least the crude product solution and the first solvent to an evaporator; and
collecting, from the evaporator, a distillate comprising the precursor compound,
wherein a purity of the precursor compound in the distillate is greater than 95% as determined by ¹H NMR.

2. The method of claim 1, wherein the crude product solution further comprises:
a second solvent,
wherein the precursor compound is dissolved in the second solvent.

3. The method of claim 2, wherein the at least one impurity is not dissolved in the second solvent.

4. The method of claim 1, wherein a purity of the precursor compound in the crude product solution is 95% or less as determined by ¹H NMR.

5. The method of claim 1, wherein a purity of the precursor compound in the crude product solution is 90% to 95% as determined by ¹H NMR.

6. The method of claim 1, wherein the precursor compound comprises a tin-containing compound.

7. The method of claim 1, wherein the precursor compound comprises a niobium-containing compound.

8. The method of claim 1, wherein the precursor compound comprises a tungsten-containing compound.

9. The method of claim 1, wherein the first solvent is inert with respect to the precursor compound.

10. The method of claim 1, wherein the boiling point of the first solvent is at least 10% greater than a boiling point of the crude product solution.

11. The method of claim 1, wherein the boiling point of the first solvent is 250° C. to 500° C.

12. The method of claim 1, wherein the boiling point of the crude product solution is 50° C. to 200° C.

13. The method of claim 1, wherein the first solvent comprises an isoparaffinic hydrocarbon fluid.

14. The method of claim 1, wherein the at least one impurity comprises at least one solid impurity.

15. The method of claim 1, wherein the at least one impurity comprises at least one of a salt compound, a dialkyl compound, or any combination thereof.

16. The method of claim 1, wherein the purity of the precursor compound in the distillate is at least 99%.

17. The method of claim 1, wherein the evaporator comprises at least one of a wiped film evaporator, a stirrer evaporator, a rising film evaporator, a circulation evaporator, a falling film evaporator, or any combination thereof.

18. The method of claim 1, further comprising:
collecting a residue comprising the at least one impurity.

19. A system comprising:
a crude product solution comprising a precursor compound and at least one impurity;
a first solvent having a boiling point that is greater than a boiling point of the crude product solution; and
an evaporator,
wherein, when a mixture of the crude product solution and the first solvent is fed to the evaporator, the evaporator is configured to produce a distillate comprising the precursor compound,
wherein a purity of the precursor compound in the distillate is greater than 95% as determined by ¹H NMR.

20. The system of claim 19, wherein the evaporator comprises at least one of wiped film evaporator, a stirrer evaporator, a rising film evaporator, a circulation evaporator, a falling film evaporator, or any combination thereof.
